(19) United States
(12) Patent Application Publication
(10) Pub. No.: US 2012/0063996 A1
(43) Pub. Date: Mar. 15, 2012

(54) NOVEL NIMESULIDE COMPOSITIONS

(75) Inventors: H. William Bosch, Bryn Mawr, PA (US); Christian F. Wertz, Landsdale, PA (US)

(73) Assignee: Elan Pharma International Ltd.

(21) Appl. No.: 13/193,545

(22) Filed: Jul. 28, 2011

Related U.S. Application Data
(63) Continuation of application No. 10/697,703, filed on Oct. 31, 2003, now abandoned.
Application No. 10/697,703 is a continuation-in-part of application No. 10/276,400, filed on Jan. 15, 2003, now abandoned.
Application No. 10/276,400 is a continuation of application No. 09/572,961, filed on May 18, 2000, now Pat. No. 6,316,029.

(51) Int. Cl. A61K 51/00; A61K 31/18; A61K 36/00; A61K 36/886; A61K 36/82; A61K 35/60; A61K 38/44; A61K 38/02; A61K 31/7052; A61P 29/00; A61P 19/02; A61P 15/00; A61P 25/06; A61P 13/00; A61P 13/08; A61P 9/00; A61P 9/10; A61P 11/02; A61P 27/16; A61P 11/06; A61P 35/00; A61P 43/00; A61P 13/12; A61P 25/28; A61P 19/08; A61P 1/12; A61P 7/02; A61P 25/08; A61P 3/04; A61P 31/10; A61P 1/08; A61P 33/10; A61P 9/06; A61P 9/12; A61P 25/24; A61P 31/04; A61P 37/08; A61P 37/06; A61P 31/12; A61P 25/22; A61P 25/20; A61P 11/14; A61K 49/00; A61K 9/14; B82Y 5/00

(52) U.S. Cl. 424/1.11; 424/400; 514/605; 424/777; 424/744; 424/729; 424/523; 424/94.4; 514/1.1; 514/52; 424/9.1; 977/773; 977/915

(57) ABSTRACT

The present invention provides nanoparticulate nimesulide compositions. The compositions preferably comprise nimesulide and at least one surface stabilizer adsorbed on or associated with the surface of the nimesulide particles. The nanoparticulate nimesulide particles preferably have an effective average particle size of less than about 2000 nm. The invention also provides methods of making and using nanoparticulate nimesulide compositions.

### RELATED APPLICATIONS

This application is a continuation of U.S. patent application Ser. No. 10/697,703, filed Oct. 31, 2003, which is a continuation-in-part of U.S. application Ser. No. 10/276,400, filed on Jan. 15, 2003, which is a continuation of U.S. application Ser. No. 09/572,961, filed on May 18, 2000, now U.S. Pat. No. 6,316,029. The contents of these applications are incorporated herein by reference in their entirety.

### FIELD OF THE INVENTION

The present invention relates to nanoparticulate compositions comprising nimesulide. The nimesulide particles in these compositions preferably have an effective average particle size of less than about 2000 nm.

### BACKGROUND OF THE INVENTION

### I. Background Regarding Nanoparticulate Active Agent Compositions

Nanoparticulate active agent compositions, first described in U.S. Pat. No. 5,145,684 (“the '684 patent”), contain particles of a poorly soluble therapeutic or diagnostic agent having adsorbed onto, or associated with, the surface thereof a non-crosslinked surface stabilizer. Such compositions provide superior bioavailability, which can be affected by factors such as dosage form and the dissolution rate of a drug. Poor bioavailability constitutes a significant problem encountered in developing of pharmaceutical compositions, particularly those containing an active ingredient that is poorly soluble in water. By decreasing the particle size of an active agent, the surface area of the composition is increased, which generally results in increased bioavailability. The '684 patent does not teach nanoparticulate compositions of nimesulide.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. Nos. 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using Iodinated Aromatic Propanedioates;” 5,336,507 for “Use of Charged Phospholipids to Reduce Nanoparticle Aggregation;” 5,340,564 for “Formulations Comprising Olin 10-G to Prevent Particle Aggregation and Increase Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,738 for “Nanoparticulate NSAID Formulations;” 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles;” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;” 5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(−)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;” 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions;” 6,316,029 for “Rapidly Disintegrating Solid Oral Dosage Form,” 6,375,986 for “Solid Dose Nanoparticulate Compositions Comprising a Synergistic Combination of a Polymeric Surface Stabilizer and Dioctyl Sodium Sulfosuccinate;” 6,428,814 for “Bioadhesive Nanoparticulate Compositions Having Cationic Surface Stabilizers;” 6,431,478 for “Small Scale Mill;” and 6,432,381 for “Methods for Targeting Drug Delivery to the Upper and/or Lower Gastrointestinal Tract,” all of which are specifically incorporated by reference. In addition, U.S. Patent Application No. 20020012675 A1, published on Jan. 31, 2002, for “Controlled Release Nanoparticulate Compositions,” and WO 02/098565 for “System and Method for Milling Materials,” describe nanoparticulate active agent compositions, and are specifically incorporated by reference. None of these references describe nanoparticulate nimesulide compositions.

Amorphous small particle compositions are described, for example, in U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” 4,997,454 for “Method for Making Uniformly-Sized Particles From Insoluble Compounds;” 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.”

### II. Background Regarding Nimesulide

Nimesulide, also known as N-(4-Nitro-2-phenoxy-phenyl)-methanesulfonamide, is a non-steroidal anti-inflammatory drug (NSAID) having the following structure:

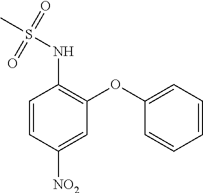

Nimesulide has a molecular weight of 308.31 g/mol and is practically insoluble in water, having a half-life of about 4 hours. It operates by selectively inhibiting cyclooxygenase-2 (COX-2).

Therapeutic concentrations of nimesulide cause several actions, including the following: (a) inhibition of prostaglandin synthesis, (b) inhibition of toxic oxygen metabolite formation, (c) inhibition of cytokine release, (d) inhibition of histamine release, and (e) inhibition of cartilage degradation. In accord with these actions, nimesulide exerts anti-inflammatory, analgesic and anti-pyretic activities, and therefore effectively treats a wide range of disorders.

One drawback of nimesulide is that it can be difficult to administer, due to its near insolubility in water. The art has addressed several ways to render nimesulide more bioavailable. U.S. Pat. No. 5,756,546 to Pirotte et al. discloses a water-soluble salt formed from equimolar amounts of nimesulide and L-lysine, but the salt can be contaminated with excess L-lysine or insoluble nimesulide upon any variation from the 1:1 molar ratio. U.S. Pat. No. 5,744,165 Geczy et al. relates to alkali and alkaline earth salts of nimesulide that, when combined with a cyclodextrin to form inclusion complexes, can be dissolved in water. However, compositions of this type can deliver unwanted amounts of cyclodextrin and sodium ion to a patient. U.S. Pat. No. 6,194,462 to Giorgetti discloses soluble formulations of nimesulide achieved by dissolving the drug in a mixture of water at basic pH and one or more alcohols such as ethanol. In contrast, U.S. Pat. No. 6,288,121 to Bader et al. discloses emulsions of nimesulide in liquid crystal form for the controlled release of the drug, while U.S. Pat. No. 5,998,480 to Giorgetti relates to bioavailable formulations of nimesulide, a phospholipid, and an organic or inorganic acid.

Nimesulide has been marketed under numerous trade names, including Ainext, Aulin®, Donulide®, Edrigyl®, Eskaflam®, Fansidol®, Flogovital®, Guaxan®, Heugan®, Mesulid®, Nemil®, Nexen®, Nide®, Nidol®, Nimed®, Nimedex®, Nisulid®, Plarium®, Scaflam®, Scaflan®, and Sulidene®.

There is a need in the art for nimesulide compositions which can decrease frequency of dosing, improve clinical efficacy, and potentially reduce side effects. The present invention satisfies these needs.

### SUMMARY OF THE INVENTION

The present invention provides nanoparticulate nimesulide compositions. The compositions preferably comprise nimesulide and at least one surface stabilizer adsorbed on or associated with the surface of the nimesulide particles. The nanoparticulate nimesulide particles preferably have an effective average particle size of less than about 2000 nm.

The invention also provides pharmaceutical compositions that comprise nanoparticulate nimesulide. The pharmaceutical compositions preferably comprise nimesulide, at least one surface stabilizer, and at least one pharmaceutically acceptable carrier, as well as any desired excipients known to those in the art. The compositions can be formulated into any desired dosage form.

In another aspect, the invention includes nanoparticulate nimesulide compositions having improved pharmacokinetic profiles, such as improved Tₘₐₓ, Cₘₐₓ, and AUC parameters, relative to conventional solubilized, microcrystalline or non-nanoparticulate nimesulide formulations.

In yet another aspect, the invention encompasses a nimesulide composition having a pharmacokinetic profile that is not substantially affected by the fed or fasted state of a subject ingesting the composition, preferably as defined by Cₘₐₓ and AUC guidelines given by the U.S. Food and Drug Administration and/or the corresponding European regulatory agency (EMEA).

Other aspects of the invention include, but are not limited to, nanoparticulate nimesulide compositions that, as compared to conventional non-nanoparticulate formulations of nimesulide, preferably have one or more of the following properties: (1) smaller dosage form size; (2) smaller doses of drug required to obtain the same pharmacological effect; (3) increased bioavailability; (4) an increased rate of dissolution for the nanoparticulate nimesulide compositions; and (5) bioadhesive nimesulide compositions.

This invention further discloses methods of making a nanoparticulate nimesulide composition. The methods comprise contacting nimesulide and at least one surface stabilizer for a time and under conditions sufficient to provide a nanoparticulate nimesulide composition. The one or more surface stabilizers can be contacted with nimesulide before, preferably during, or after size reduction of the nimesulide.

The present invention also includes methods of using nanoparticulate nimesulide compositions for treating a wide range of conditions and disorders mediated by COX-2, including, but not limited to, disorders characterized by inflammation, pain, and/or fever. Thus, compositions of the invention are useful for indications where anti-inflammatory agents, anti-angiogenesis agents, antitumorigenic agents, immunosuppressive agents, NSAIDs, COX-2 inhibitors, analgesic agents, anti-thrombotic agents, narcotic or antifebrile agents are typically used.

The methods comprise administering to a subject a therapeutically effective amount of a nanoparticulate nimesulide pharmaceutical composition according to the invention. Additionally, a subject may be administered a therapeutic amount of a pharmaceutical composition that comprises both nanoparticulate nimesulide and non-nanoparticulate nimesulide. Alternatively, the methods comprise administering to a subject a therapeutically effective amount of a nanoparticulate nimesulide composition in combination with one or more non-nimesulide active agents.

Both the foregoing general description and the following detailed description are exemplary and explanatory, and are intended to provide further explanation of the invention as claimed. Other objects, advantages, and novel features will be readily apparent to those skilled in the art from the following detailed description of the invention.

### DETAILED DESCRIPTION OF THE INVENTION

The present invention relates to nanoparticulate nimesulide compositions. The compositions preferably comprise nimesulide and at least one surface stabilizer adsorbed on or associated with the surface of the nimesulide particles. The nanoparticulate nimesulide particles preferably have an effective average particle size of less than about 2000 nm.

The present invention also relates to pharmaceutical compositions that comprise a nanoparticulate nimesulide active agent. The pharmaceutical compositions preferably comprise nimesulide, at least one surface stabilizer, and at least one pharmaceutically acceptable carrier. Non-toxic physiologically acceptable carriers, adjuvants, and vehicles are collectively referred to as carriers herein.

The pharmaceutical compositions may be formulated for parenteral injection, including intravenous, intramuscular, or subcutaneous; oral administration in solid, liquid, or aerosol form; or vaginal, nasal, rectal, ocular, local (such as in powders, ointments or drops), buccal, intracisternal, intraperitoneal, or topical administration, and the like.

Solid dosage forms are preferred, though any pharmaceutically acceptable dosage form may be employed. Exemplary solid dosage forms include, but are not limited to, tablets, capsules, sachets, lozenges, powders, pills, or granules. A solid dosage form may be, for example, a fast melt dosage form, controlled release dosage form, lyophilized dosage form, delayed release dosage form, extended release dosage form, pulsatile release dosage form, mixed immediate release and controlled release dosage form, or a combination thereof. Solid dose tablet formulations are preferred.

### I. Technical Challenges Overcome by the Inventors

As taught in the '684 patent, not every combination of surface stabilizer and active agent will result in a stable nanoparticulate composition. Thus, the discovery that stable nanoparticulate nimesulide formulations can be made was surprising.

In general, the rate of dissolution of a particulate drug increases with increasing surface area, e.g., decreasing particle size. Consequently, methods of making finely divided drugs have been studied and efforts have been made to control the size and size range of drug particles in pharmaceutical compositions. Nanoparticulate active agent formulations suitable for administration as pharmaceuticals require formulation of the active ingredient into a colloidal dispersion that exhibits the acceptable nanoparticle size range and the stability to maintain such size range without agglomerating. Thus, merely increasing surface area by decreasing particle size does not assure success. Further challenges include forming solid dose forms that are redispersible into nanoparticle form upon administration to a patient, to maintain the benefit of nanoparticle nimesulide over a traditional microparticulate or solubilized nimesulide dosage form.

### II. Summary of Advantages of Nanoparticulate Nimesulide Formulations

Advantages of nanoparticulate nimesulide formulations, relative to conventional non-nanoparticulate or solubilized formulations of nimesulide include, but are not limited to: (1) faster onset of action; (2) a potential decrease in the frequency of dosing; (3) smaller tablet (or other solid dosage form) size or liquid dose volumes; (4) smaller doses of drug required to obtain the same pharmacological effect; (5) increased bioavailability; (6) an increased rate of dissolution; (7) high redispersibility of the nanoparticulate nimesulide particles present in the compositions of the invention following administration; (8) improved performance characteristics for oral, intravenous, subcutaneous, or intramuscular injection, such as higher dose loading; (9) improved pharmacokinetic profiles, such as improved Tₘₐₓ, Cₘₐₓ, and AUC profiles; (10) substantially similar or bioequivalent pharmacokinetic profiles of the nanoparticulate nimesulide compositions when administered in the fed versus the fasted state; (11) bioadhesive nimesulide compositions; (12) low viscosity liquid nanoparticulate nimesulide dosage forms can be made; (13) for liquid nanoparticulate nimesulide compositions having a low viscosity—better subject compliance due to the perception of a lighter formulation which is easier to consume and digest; (14) for liquid nanoparticulate nimesulide compositions having a low viscosity—ease of dispensing because one can use a cup or a syringe; (15) the nanoparticulate nimesulide compositions can be sterile filtered; (16) the nanoparticulate nimesulide compositions can be used in conjunction with other active agents; (17) the nanoparticulate nimesulide compositions are suitable for parenteral administration; and (18) the nanoparticulate nimesulide compositions do not require organic solvents or pH extremes.

Moreover, nanoparticulate nimesulide formulations do not possess the sedative and addictive properties of narcotic analgesics. Because nimesulide does not cause drowsiness and is not addictive, it is a preferred analgesic when ambulation is important or when treatment is protracted and chemical dependency could result from prolonged use of narcotic analgesics.

### III. DEFINITIONS

The present invention is described herein using several definitions that are set forth below and throughout the specification.

“About” will be understood by persons of ordinary skill in the art and will vary to some extent on the context in which the term is used. If there are uses of the term that are not clear to persons of ordinary skill in the art given the context in which it is used, “about” will mean up to plus or minus 10% of the particular term.

“Conventional” or “non-nanoparticulate active agent” means an active agent that is solubilized or that has an effective average particle size of greater than about 2 microns. “Effective average particle size of greater than about 2 microns” means that at least 50% of the particles of the composition have a size of greater than about 2 microns.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salts” as used herein refers to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Poorly water soluble drugs” as used herein means drugs having a solubility of less than about 30 mg/ml, preferably less than about 20 mg/ml, preferably less than about 10 mg/ml, or preferably less than about 1 mg/ml. Such drugs tend to be eliminated from the gastrointestinal tract before being absorbed into the circulation. Moreover, poorly water soluble drugs tend to be unsafe for intravenous administration techniques, which are used primarily in conjunction with highly water soluble drug substances.

As used herein with reference to stable nimesulide particles, “stable” includes, but is not limited to, one or more of the following parameters: (1) that the nimesulide particles do not appreciably flocculate or agglomerate due to interparticle attractive forces, or otherwise significantly increase in particle size over time; (2) that the physical structure of the nimesulide particles is not altered over time, such as by conversion from an amorphous phase to crystalline phase; (3) that the nimesulide particles are chemically stable; and/or (4) where the nimesulide has not been subject to a heating step at or above the melting point of the nimesulide in the preparation of the nanoparticles of the invention.

“Therapeutically effective amount” as used herein with respect to a drug dosage, means a dosage that provides the specific pharmacological response for which the drug is administered in a significant number of subjects in need of such treatment. A “therapeutically effective amount,” administered to a particular subject in a particular instance, will not always effectively treat the diseases described herein, even though such dosage is deemed a ‘therapeutically effective amount’ by those skilled in the art. Throughout this description, drug dosages are, in particular instances, measured as oral dosages, or with reference to drug levels as measured in blood.

### IV. Preferred Characteristics of Nanoparticulate Nimesulide Compositions

A. Fast Onset of Activity

Of particular importance, conventional formulations of nimesulide are inappropriate for managing acute pain due to delayed onset of action, as conventional nimesulide formulations have a Tₘₐₓ of 4-6 hours, which is more than five times as long as most narcotic analgesic drugs. See The Physician's Desk Reference, 56^{th} Ed., pp. 446 and 1054. Unlike conventional nimesulide formulations, nanoparticulate nimesulide formulations, which exhibit faster onset of action, are useful for treating acute pain where fast pain relief is required.

B. Increased Bioavailability and Lower Dosages

Relative to conventional nimesulide formulations, the inventive nanoparticulate nimesulide compositions preferably exhibit increased bioavailability, require smaller doses, and show longer plasma half-life.

As another advantage, nanoparticulate formulations of nimesulide also provide a longer duration of pain relief relative to traditional narcotic analgesic drugs. While traditional narcotics provide fast onset of action, the duration of pain relief is short. Nanoparticulate nimesulide formulations combine the fast onset of traditional narcotics with the duration of pain relief of conventional NSAIDs. The long half-life of nimesulide, approximately 20 hours as compared to 2-3 hours for most narcotics, confers a long duration of action and thus requires less frequent dosing.

Enhanced bioavailability enables the use of lower doses, which also results in decreased toxicity associated with nimesulide. In this regard, lower doses of nanoparticulate nimesulide can achieve the same or better therapeutic effects as larger doses of conventional nimesulide. Such lower doses can be realized due to the greater bioavailability of nanoparticulate drug formulations as compared to conventional drug formulations. Nimesulide, like any drug, can have adverse side effects. Therefore, the ability to administer lower doses of it translates into fewer adverse side effects.

Enhanced bioavailability also can enable the use of a smaller dosage size. This is significant for certain patient populations, such as the elderly, juvenile and infant.

C. Improved Pharmacokinetic Profiles

The inventive nanoparticulate nimesulide compositions also preferably exhibit a desirable pharmacokinetic profile when administered to mammalian subjects. The desirable pharmacokinetic profile preferably includes, but is not limited to: (1) that the Tₘₐₓ of nimesulide, when assayed in the plasma of a mammalian subject following administration, is preferably less than the Tₘₐₓ for a conventional, non-nanoparticulate form of nimesulide administered at the same dosage; (2) that the Cₘₐₓ of nimesulide, when assayed in the plasma of a mammalian subject following administration, is preferably greater than the Cₘₐₓ for a conventional, non-nanoparticulate form of nimesulide administered at the same dosage; and/or (3) that the AUC of nimesulide, when assayed in the plasma of a mammalian subject following administration, is preferably greater than the AUC for a conventional, non-nanoparticulate form of nimesulide administered at the same dosage.

The desirable pharmacokinetic profile, as used herein, is the pharmacokinetic profile measured after an initial dose of nimesulide. The dose can be formulated in any way as described below and as known to those skilled in the art.

A preferred nanoparticulate nimesulide composition exhibits, in comparative pharmacokinetic testing with a non-nanoparticulate formulation of nimesulide administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, or not greater than about 10% of the Tₘₐₓ, exhibited by the non-nanoparticulate formulation of nimesulide.

A preferred nanoparticulate nimesulide composition exhibits, in comparative pharmacokinetic testing with a non-nanoparticulate formulation of nimesulide administered at the same dosage, a Cₘₐₓ that is at least about 10%, at least about 20%, at least about 30%, at least about 40%, at least about 50%, at least about 60%, at least about 70%, at least about 80%, at least about 90%, or at least about 100% greater than the Cₘₐₓ exhibited by the non-nanoparticulate formulation of nimesulide.

A preferred nanoparticulate nimesulide composition exhibits, in comparative pharmacokinetic testing with a non-nanoparticulate formulation of nimesulide administered at the same dosage, an AUC that is at least about 10%, at least about 20%, at least about 30%, at least about 40%, at least about 50%, at least about 60%, at least about 70%, at least about 80%, at least about 90%, or at least about 100% greater than the AUC exhibited by the non-nanoparticulate formulation of nimesulide.

According to the invention, any formulation that provides the desired pharmacokinetic profile is suitable for administration. Exemplary types of formulations that give such profiles are liquid dispersions, gels, aerosols, ointments, creams and solid dose forms.

D. The Pharmacokinetic Profiles are not Affected by the Fed or Fasted State of the Subject Ingesting the Compositions

Certain drugs have been shown to have significantly lower plasma levels when administered under fasting conditions as when administered immediately after a standard test meal. This significant difference is undesirable.

Nanoparticulate nimesulide formulations of the invention preferably alleviate this problem. That is, they preferably reduce the differences in, or more preferably do not produce significantly different, absorption levels when administered under fed as compared to fasting conditions.

Thus, the invention encompasses a nimesulide composition having a pharmacokinetic profile that is not substantially affected by the fed or fasted state of a subject ingesting the composition. This means that there is no substantial difference in the quantity of drug absorbed or the rate of drug absorption when the nanoparticulate nimesulide compositions are administered in the fed versus the fasted state.

The invention also encompasses a nimesulide composition for which administration to a subject in a fasted state is bioequivalent to administration to a subject in a fed state. “Bioequivalency” is preferably established by a 90% Confidence Interval (CI) of between 0.80 and 1.25 for both Cₘₐₓ and AUC under U.S. Food and Drug Administration regulatory guidelines, or a 90% CI for AUC of between 0.80 to 1.25 and a 90% CI for Cₘₐₓ of between 0.70 to 1.43 under the European EMEA regulatory guidelines (Tₘₐₓ is not relevant for bioequivalency determinations under USFDA and EMEA regulatory guidelines).

Benefits of a dosage form that substantially eliminates the effect of food include an increase in convenience, which increases patient compliance, as a patient does not need to ensure that they are taking a dose either with or without food. This is significant, as poor patient compliance can defeat the purpose of administering a drug.

The difference in absorption of the inventive nimesulide compositions, when administered in a fed versus a fasted state, preferably is less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, or less than about 3%.

E. Rapid Dissolution Profiles

Nanoparticulate nimesulide compositions of the invention preferably have rapid dissolution profiles. Rapid dissolution of an administered active agent is desirable, as faster dissolution generally leads to faster onset of action and greater bioavailability. To maximize the dissolution profile and bioavailability of nimesulide, it would be useful to increase the drug's dissolution so that it could attain a level close to 100%.

The inventive nimesulide compositions preferably have a dissolution profile in which at least about 20% of the composition is dissolved within 5 minutes. In other embodiments, at least about 30% or about 40% of the nimesulide composition is dissolved within about 5 minutes. In yet other embodiments, preferably at least about 40%, about 50%, about 60%, about 70%, or about 80% of the nimesulide composition is dissolved within about 10 minutes. Finally, in another embodiment, preferably at least about 70%, about 80%, about 90%, or about 100% of the nimesulide composition is dissolved within about 20 minutes.

Dissolution is preferably measured in a medium that is discriminating. Such a dissolution medium will produce two different dissolution curves for two products having different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be performed by spectrophotometry. The rotating blade method (European Pharmacopoeia) can be used to measure dissolution.

F. Redispersion of Nanoparticulate Nimesulide Dosage Forms to Nanoparticulate Particle Size

The inventive nanoparticulate nimesulide compositions preferably redisperse such that the effective average particle size of the redispersed nimesulide particles is less than about 2 microns. This is significant because if, upon administration, the nanoparticulate nimesulide compositions did not redisperse to a substantially nanoparticulate particle size, then the dosage form might lose the benefits of a nanoparticulate formulation.

This is because nanoparticulate active agent compositions benefit from the small particle size of the active agent. If the active agent does not redisperse into small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall well below that observed with a liquid dispersion form of the nanoparticulate active agent.

Moreover, the inventive nanoparticulate nimesulide compositions preferably exhibit dramatic redispersion upon administration to a mammal, such as a human or animal. This can be demonstrated by reconstitution/redispersion in a biorelevant aqueous medium such that the effective average particle size of the redispersed nimesulide particles is less than about 2 microns. Biorelevant aqueous media include any aqueous media that exhibit representative ionic strength and pH. The desired pH and ionic strength is one that represents physiological conditions found in the human body. Such biorelevant aqueous media can be, for example, aqueous electrolyte solutions or aqueous solutions of any salt, acid, base, or a combination thereof.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M, while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of a test solution are more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, that exhibit the desired pH and ionic strength include, but are not limited to, phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed nimesulide particles (redispersed in an aqueous, biorelevant, or any other suitable media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the nimesulide particles have a particle size less than the effective average, by weight, i.e., less than about 2000 nm, 1900 nm, 1800 nm, etc., when measured by the above-noted techniques. Preferably, at least about 70%, about 90%, about 95%, or about 99% of the nimesulide particles have a particle size less than the effective average, i.e., less than about 2000 nm, 1900 nm, 1800 nm, 1700 nm, etc.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

G. Bioadhesive Nanoparticulate Nimesulide Compositions

Nanoparticulate nimesulide compositions of the invention can exhibit bioadhesive properties. Such compositions comprise one or more cationic surface stabilizers, which are described in more detail below.

The term bioadhesion refers to any attractive interaction between two biological surfaces or between a biological and a synthetic surface. In the case of bioadhesive nanoparticulate nimesulide compositions, the term bioadhesion describes the adhesion between nanoparticulate nimesulide compositions and a biological substrate (e.g., gastrointestinal mucin, lung tissue, nasal mucosa, etc.). See, e.g., U.S. Pat. No. 6,428,814 for “Bioadhesive Nanoparticulate Compositions Having Cationic Surface Stabilizers,” which is specifically incorporated by reference. Bioadhesive formulations of nimesulide exhibit exceptional bioadhesion to biological substrates.

Bioadhesive nimesulide compositions are useful in any situation where it is desirable to apply the compositions to a biological surface. Bioadhesive nimesulide compositions coat the targeted surface in a continuous and uniform film, which is invisible to the naked human eye.

Bioadhesion slows transit of a nimesulide composition, and some nimesulide particles inevitably adhere to tissue other than the mucous cells. This provides a prolonged exposure to nimesulide, thereby increasing absorption and bioavailability of the administered dosage.

H. Low Viscosity Liquid Nanoparticulate Nimesulide Compositions

A liquid dosage form of a conventional microcrystalline or non-nanoparticulate or solubilized nimesulide composition would be expected to be a relatively large volume, highly viscous substance which would not be well accepted by patient populations. This is significant, as liquid dosage forms can be particularly useful for patient populations such as the elderly and infant.

Liquid dosage forms of the nanoparticulate nimesulide compositions of the invention provide significant advantages over a liquid dosage form of a conventional microcrystalline or solubilized nimesulide composition. The low viscosity and silky texture of liquid dosage forms of the nanoparticulate nimesulide compositions of the invention result in advantages in both preparation and use. These advantages include, for example: (1) better subject compliance due to the perception of a lighter formulation which is easier to injest; (2) ease of dispensing as compared to a highly viscous formulation; (3) potential for formulating a higher concentration of nimesulide resulting in a smaller dosage volume and thus less volume for the subject to consume; and (4) easier overall formulation concerns.

The viscosities of liquid dosage forms of nanoparticulate nimesulide according to the invention are preferably less than about 1/200, less than about 1/175, less than about 1/150, less than about 1/125, less than about 1/100, less than about 1/75, less than about 1/50, or less than about 1/25 of a topical liquid dosage form of a non-nanoparticulate nimesulide composition, at about the same concentration per ml of nimesulide.

Typically the viscosity of liquid nanoparticulate nimesulide dosage forms of the invention, at a shear rate of 0.1 (1/s) and measured at 20° C., is from about 2000 mPa·s to about 1 mPa·s, from about 1900 mPa·s to about 1 mPa·s, from about 1800 mPa·s to about 1 mPa·s, from about 1700 mPa·s to about 1 mPa·s, from about 1600 mPa·s to about 1 mPa·s, from about 1500 mPa·s to about 1 mPa·s, from about 1400 mPa·s to about 1 mPa·s, from about 1300 mPa·s to about 1 mPa·s, from about 1200 mPa·s to about 1 mPa·s, from about 1100 mPa·s to about 1 mPa·s, from about 1000 mPa·s to about 1 mPa·s, from about 900 mPa·s to about 1 mPa·s, from about 800 mPa·s to about 1 mPa·s, from about 700 mPa·s to about 1 mPa·s, from about 600 mPa·s to about 1 mPa·s, from about 500 mPa·s to about 1 mPa·s, from about 400 mPa·s to about 1 mPa·s, from about 300 mPa·s to about 1 mPa·s, from about 200 mPa·s to about 1 mPa·s, from about 175 mPa·s to about 1 mPa·s, from about 150 mPa·s to about 1 mPa·s, from about 125 mPa·s to about 1 mPa·s, from about 100 mPa·s to about 1 mPa·s, from about 75 mPa·s to about 1 mPa·s, from about 50 mPa·s to about 1 mPa·s, from about 25 mPa·s to about 1 mPa·s, from about 15 mPa·s to about 1 mPa·s, from about 10 mPa·s to about 1 mPa·s, or from about 5 mPa·s to about 1 mPa·s.

Viscosity is concentration and temperature dependent. Typically, a higher concentration results in a higher viscosity, while a higher temperature results in a lower viscosity. Viscosity as defined above refers to measurements taken at about 20° C. (The viscosity of water at 20° C. is 1 mPa·s.) The invention encompasses equivalent viscosities measured at different temperatures.

The liquid formulations of this invention can be formulated for dosages in any volume but preferably equivalent or smaller volumes than a liquid dosage form of a non-nanoparticulate or solubilized nimesulide composition.

I. Sterile Filtered Nanoparticulate Nimesulide Compositions

The nanoparticulate nimesulide compositions of the invention can be sterile filtered. This obviates the need for heat sterilization, which can harm or degrade nimesulide, as well as result in crystal growth and particle aggregation.

Sterile filtration can be difficult because of the required small particle size of the composition. Filtration is an effective method for sterilizing homogeneous solutions when the membrane filter pore size is less than or equal to about 0.2 microns (200 nm) because a 0.2 micron filter is sufficient to remove essentially all bacteria. Sterile filtration is normally not used to sterilize suspensions of micron-sized nimesulide because the nimesulide particles are too large to pass through the membrane pores.

A sterile nanoparticulate nimesulide dosage form is particularly useful in treating immunocompromised patients, infants or juvenile patients, and the elderly, as these patient groups are the most susceptible to infection caused by a non-sterile liquid dosage form.

Because the nanoparticulate nimesulide compositions of the invention, formulated into a liquid dosage form, can be sterile filtered, and because the compositions can have a very small nimesulide effective average particle size, the compositions are suitable for parenteral administration.

J. Combination Pharmacokinetic Profile Compositions

In one embodiment of the invention, a first nimesulide formulation providing the pharmacokinetic profile described above is co-administered with at least one other nimesulide formulation that generates a different pharmacokinetic profile, specifically one exhibiting slower absorption into the bloodstream, and therefore a longer Tₘₐₓ and typically a lower Cₘₐₓ. For example, the second nimesulide formulation can have a conventional particle size, which produces a longer Tₘₐₓ, and typically a lower Cₘₐₓ. Alternatively, a second, third or fourth nimesulide formulation can differ from the first, and from each other, in the effective average particle sizes of each composition. The difference particle sizes produce different Tₘₐₓ values. The combination of fast pain relief provided by the first formulation and longer-lasting pain relief provided by the second (or third, fourth, etc.) formulation can reduce the dose frequency required.

If the second nimesulide composition has a nanoparticulate particle size, then preferably the nimesulide particles of the second composition have at least one surface stabilizer associated with the surface of the drug particles. The one or more surface stabilizers can be the same as or different from the surface stabilizer(s) present in the first nimesulide composition.

Preferably where co-administration of a “fast-acting” formulation and a “longer-lasting” formulation is desired, the two formulations are combined within a single composition, for example a dual-release composition.

K. Combination Active Agent Compositions

The invention encompasses the nanoparticulate nimesulide compositions of the invention formulated or co-administered with one or more non-nimesulide active agents. Methods of using such combination compositions are also encompassed by the invention. The non-nimesulide active agents can be present in a crystalline phase, an amorphous phase, a semi-crystalline phase, a semi-amorphous phase, or a mixture thereof.

The compound to be administered in combination with a nanoparticulate nimesulide composition of the invention can be formulated separately from the nanoparticulate nimesulide composition or co-formulated with the nanoparticulate nimesulide composition. Where a nanoparticulate nimesulide composition is co-formulated with a second active agent, the second active agent can be formulated in any suitable manner, such as immediate-release, rapid-onset, sustained-release, or dual-release form.

Such non-nimesulide active agents can be, for example, a therapeutic agent. A therapeutic agent can be a pharmaceutical agent, including a biologic, such as proteins, peptides, and nucleotides, or a diagnostic agent, such as a contrast agent, including x-ray contrast agents. The active agent can be selected from a variety of known classes of drugs, including, for example, amino acids, proteins, peptides, nucleotides, anti-obesity drugs, central nervous system stimulants, carotenoids, corticosteroids, elastase inhibitors, anti-fungals, oncology therapies, anti-emetics, analgesics, cardiovascular agents, anti-inflammatory agents, such as NSAIDs and COX-2 inhibitors, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives (hypnotics and neuroleptics), astringents, alpha-adrenergic receptor blocking agents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, vasomodulators, and xanthines.

Examples of representative active agents useful in this invention include, but are not limited to, acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, glipizide, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, and acetylsalicylate.

A description of these classes of active agents and a listing of species within each class can be found in Martindale's The Extra Pharmacopoeia, 31^{st} Edition (The Pharmaceutical Press, London, 1996), specifically incorporated by reference. The active agents are commercially available and/or can be prepared by techniques known in the art.

Exemplary nutraceuticals or dietary supplements include, but are not limited to, lutein, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., arginine, iso-leucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as “pharmafoods.”

Exemplary nutraceuticals and dietary supplements are disclosed, for example, in Roberts et al., Nutraceuticals: The Complete Encyclopedia of Supplements, Herbs, Vitamins, and Healing Foods (American Nutraceutical Association, 2001), which is specifically incorporated by reference. Dietary supplements and nutraceuticals are also disclosed in Physicians' Desk Reference for Nutritional Supplements, 1st Ed. (2001) and The Physicians' Desk Reference for Herbal Medicines, 1st Ed. (2001), both of which are also incorporated by reference. A nutraceutical or dietary supplement, also known as a phytochemical or functional food, is generally any one of a class of dietary supplements, vitamins, minerals, herbs, or healing foods that have medical or pharmaceutical effects on the body.

Preferred combination therapies comprise a composition useful in methods of the invention with one or more compounds selected from aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylori, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen, and zomepirac. See The Merck Index, 12th Edition (1996), Therapeutic Category and Biological Activity Index, lists therein headed “Analgesic”, “Anti-inflammatory”, and “Antipyretic”).

In a particularly preferred embodiment of the invention, the nanoparticulate nimesulide composition is combined with at least one analgesic. Useful analgesics include, for example, NSAIDS and non-nimesulide COX-2 inhibitors.

Particularly preferred combination therapies comprise use of a nanoparticulate nimesulide composition of the invention with an opioid compound, more particularly where the opioid compound is codeine, meperidine, morphine, or a derivative thereof.

In an embodiment of the invention, particularly where a COX-2 mediated condition is headache or migraine, the nanoparticulate nimesulide composition is administered in combination therapy with a vasomodulator, preferably a xanthine derivative having vasomodulatory effect, more preferably an alkylxanthine compound.

Combination therapies wherein an alkylxanthine compound is co-administered with a nanoparticulate nimesulide composition as provided herein are embraced by the present embodiment of the invention whether or not the alkylxanthine is a vasomodulator and whether or not the therapeutic effectiveness of the combination is to any degree attributable to a vasomodulatory effect. The term “alkylxanthine” herein embraces xanthine derivatives having one or more C₁₋₄ alkyl substituents, preferably methyl, and pharmaceutically acceptable salts of such xanthine derivatives. Dimethylxanthines and trimethylxanthines, including caffeine, theobromine, and theophylline, are especially preferred. Most preferably, the alkylxanthine compound is caffeine.

Exemplary NSAIDS that can be formulated in combination with the nanoparticulate nimesulide composition of the invention include, but are not limited to, suitable nonacidic and acidic compounds. Suitable nonacidic compounds include, for example, nabumetone, tiaramide, proquazone, bufexamac, flumizole, epirazole, tinoridine, timegadine, and dapsone.

Exemplary acidic NSAID compounds that can be formulated in combination with the nanoparticulate nimesulide composition of the invention include, but are not limited to, carboxylic acids and enolic acids. Suitable carboxylic acid NSAIDs include, for example: (1) salicylic acids and esters thereof, such as aspirin, diflunisal, benorylate, and fosfosal; (2) acetic acids, including phenylacetic acids such as diclofenac, alclofenac and fenclofenac; (3) carbo- and heterocyclic acetic acids such as etodolac, indomethacin, sulindac, tolmetin, fentiazac, and tilomisole; (4) propionic acids, such as carprofen, fenbufen, flurbiprofen, ketoprofen, oxaprozin, suprofen, tiaprofenic acid, ibuprofen, naproxen, fenoprofen, indoprofen, pirprofen; and (5) fenamic acids, such as flufenamic, mefenamic, meclofenamic and niflumic. Suitable enolic acid NSAIDs that can be formulated in combination with the nanoparticulate nimesulide composition of the invention include, but are not limited to: (1) pyrazolones such as oxyphenbutazone, phenylbutazone, apazone, and feprazone; and (2) oxicams such as piroxicam, sudoxicam, isoxicam, and tenoxicam.

Exemplary COX-2 inhibitors that can be formulated in combination with the nanoparticulate nimesulide composition of the invention include, but are not limited to, celecoxib (SC-58635, CELEBREX®, Pharmacia/Searle & Co.), rofecoxib (MK-966, L-748731, VIOXX®, Merck & Co.), meloxicam (MOBIC®, co-marketed by Abbott Laboratories, Chicago, Ill., and Boehringer Ingelheim Pharmaceuticals), valdecoxib (BEXTRA®, G. D. Searle & Co.), parecoxib (G. D. Searle & Co.), etoricoxib (MK-663; Merck), SC-236 (chemical name of 4-[5-(4-chlorophenyl)-3-(trifluoromethyl)-1H-pyrazol-1-yl)]benzenesulfonamide; G. D. Searle & Co., Skokie, Ill.); NS-398 (N-(2-cyclohexyloxy-4-nitrophenyl)methane sulfonamide; Taisho Pharmaceutical Co., Ltd., Japan); SC-58125 (methyl sulfone spiro(2.4)hept-5-ene I; Pharmacia/Searle & Co.); SC-57666 (Pharmacia/Searle & Co.); SC-558 (Pharmacia/Searle & Co.); SC-560 (Pharmacia/Searle & Co.); etodolac (Lodine®, Wyeth-Ayerst Laboratories, Inc.); DFU (5,5-dimethyl-3-(3-fluorophenyl)-4-(4-methylsulfonyl)phenyl 2(5H)-furanone); monteleukast (MK-476), L-745337 ((5-methanesulphonamide-6-(2,4-difluorothio-phenyl)-1-indanone), L-761066, L-761000, L-748780 (all Merck & Co.); DUP-697 (5-Bromo-2-(4-fluorophenyl)-3-(4-(methylsulfonyl)phenyl; DuPont Merck Pharmaceutical Co.); PGV 20229 (1-(7-tert.-butyl-2,3-dihydro-3,3-dimethylbenzo(b)furan-5-yl)-4-cyclopropylbutan-1-one; Procter & Gamble Pharmaceuticals); iguratimod (T-614; 3-formylamino-7-methylsulfonylamino-6-phenoxy-4H-1-benzopyran-4-one; Toyama Corp., Japan); BF 389 (Biofor, USA); CL 1004 (PD 136095), PD 136005, PD 142893, PD 138387, and PD 145065 (all Parke-Davis/Warner-Lambert Co.); flurbiprofen (ANSAID; Pharmacia & Upjohn); nabumetone (FELAFEN®; SmithKline Beecham, plc); flosulide (CGP 28238; Novartis/Ciba Geigy); piroxicam (FELDANE®; Pfizer); diclofenac (VOLTAREN® and CATAFLAM®, Novartis); lumiracoxib (COX-189; Novartis); D 1367 (Celltech Chiroscience, plc); R 807 (3 benzoyldifluoromethane sulfonanilide, diflumidone); JTE-522 (Japan Tobacco, Japan); FK-3311 (4′-Acetyl-2′-(2,4-difluorophenoxy)methanesulfonanilide), FK 867, FR 140423, and FR 115068 (all Fujisawa, Japan); GR 253035 (Glaxo Wellcome); RWJ 63556 (Johnson & Johnson); RWJ 20485 (Johnson & Johnson); ZK 38997 (Schering); S 2474 ((E)-(5)-(3,5-di-tert-butyl-4-hydroxybenzylidene)-2-ethyl-1,2-isothiazolidine-1,1-dioxide indomethacin; Shionogi & Co., Ltd., Japan); zomepirac analogs, such as RS 57067 and RS 104897 (Hoffmann La Roche); RS 104894 (Hoffmann La Roche); SC 41930 (Monsanto); pranlukast (SB 205312, Ono-1078, ONON®, ULTAIR®; SmithKline Beecham); SB 209670 (SmithKline Beecham); and APHS (heptinylsulfide).

L. Miscellaneous Benefits of the Nanoparticulate Nimesulide Compositions of the Invention

The nanoparticulate nimesulide compositions preferably exhibit an increased rate of dissolution as compared to microcrystalline or non-nanoparticulate forms of nimesulide. In addition, the nanoparticulate nimesulide compositions preferably exhibit improved performance characteristics for oral, intravenous, subcutaneous, or intramuscular injection, such as higher dose loading and smaller tablet or liquid dose volumes. Moreover, the nanoparticulate nimesulide compositions of the invention do not require organic solvents or pH extremes.

### V. Compositions

The present invention includes nanoparticulate active agent compositions that comprise nimesulide. The compositions preferably comprise nimesulide and at least one surface stabilizer adsorbed on, or associated with, the surface of the nimesulide. The nanoparticulate nimesulide particles preferably have an effective average particle size of less than about 2000 nm. In another aspect, the invention provides novel combinations of nimesulide and other active agents.

The invention also provides nanoparticulate nimesulide compositions together with one or more non-toxic physiologically acceptable carriers, adjuvants, or vehicles, collectively referred to as carriers. The compositions can be formulated for various routes of administration including, but not limited to, oral, vaginal, rectal, nasal, ocular, parenteral injection (e.g., intravenous, intramuscular, or subcutaneous), local (e.g., in powder, ointment or drop form), buccal, intracisternal, intraperitoneal, or topical administration, and the like.

A. Nimesulide Particles

As used herein “nimesulide” means N-(4-Nitro-2-phenoxy-phenyl)-methanesulfonamide, which has the following structure:

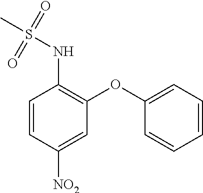

or an analog or salt thereof. The nimesulide may be in a crystalline phase, an amorphous phase, a semi-crystalline phase, a semi-amorphous phase, or a mixture thereof.

Nanoparticulate nimesulide compositions are contemplated to be useful for treating and/or preventing a wide range of conditions and disorders mediated by COX-2, including but not limited to, disorders characterized by inflammation, pain, and/or fever. Such compositions are especially useful as anti-inflammatory agents, such as in treatment of arthritis, with the additional benefit of having significantly less harmful side effects than compositions of conventional NSAIDs that lack selectivity for COX-2 over COX-1. In particular, such compositions have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs.

Thus, nanoparticulate nimesulide compositions of the invention are particularly useful as an alternative to conventional NSAIDs where such NSAIDs are contraindicated, for example in patients with peptic ulcers, gastritis, regional enteritis, ulcerative colitis, diverticulitis, or with a recurrent history of gastrointestinal lesions; gastrointestinal bleeding; coagulation disorders including anemia such as hypoprothrombinemia, hemophilia, or other bleeding problems; kidney disease; or in patients prior to surgery or patients taking anticoagulants.

Because of the rapid onset of therapeutic effect observed with the compositions of the invention, these compositions have particular advantages over prior conventional formulations for treatment of acute COX-2 mediated disorders, especially for relief of pain, for example in headache, including sinus headache and migraine.

Nimesulide is also useful in treating and/or preventing, for example, arthritic disorders, gastrointestinal conditions, inflammatory conditions, pulmonary inflammation, opthalmic diseases, central nervous systems disorders, pain, inflammation-related cardiovascular disorders, angiogenesis-related disorders, benign and malignant tumors, adenomatous polyps, disorders of the female reproductive system such as endometriosis, osteoporosis, dysmenorrhea, premature labor, asthma, eosinophil-related disorders, pyrexia, bone resorption, nephrotoxicity, hypotension, arthrosis, joint stiffness, kidney disease, liver disease including hepatitis, acute mastitis, diarrhea, colonic adenomas, bronchitis, allergic neuritis, cytomegalovirus infectivity, apoptosis including HIV-induced apoptosis, lumbago; skin-related conditions such as psoriasis, eczema, acne, burns, dermatitis, and ultraviolet radiation damage including sunburn; allergic rhinitis, respiratory distress syndrome, and endotoxin shock syndrome. Nanoparticulate nimesulide is also useful as an immunosuppressive agent.

Exemplary forms of arthritic disorders that can be treated include, but are not limited to, osteoarthritis, rheumatoid arthritis, spondyloarthropathies, gouty arthritis, juvenile arthritis, gout, ankylosing spondylitis, systemic lupus erythematosus, bursitis, tendinitis, myofascial pain, carpal tunnel syndrome, fibromyalgia syndrome, infectious arthritis, psoriatic arthritis, Reiter's syndrome, and scleroderma

Exemplary gastrointestinal conditions or ulcerative diseases that can be treated include, but are not limited to, inflammatory bowel disease, Crohn's disease, gastritis, irritable bowel syndrome, ulcerative colitis, gastric ulcer, pathological but non-malignant conditions such as hemangiomas, including infantile hemaginomas, angiofibroma of the nasopharynx, and avascular necrosis of bone.

Exemplary inflammation conditions that can be treated include, but are not limited to, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, post-operative inflammation including that following ophthalmic surgery such as cataract surgery or refractive surgery, and the like.

Exemplary pulmonary inflammation conditions that can be treated include, but are not limited to, inflammation associated with viral infections and cystic fibrosis, and in bone resorption such as that associated with osteoporosis.

Exemplary opthalmic diseases or conditions that can be treated include, but are not limited to, retinitis, conjunctivitis, retinopathies, uveitis, ocular photophobia, acute injury to the eye tissue, corneal graft rejection, ocular neovascularization, retinal neovascularization including neovascularization following injury or infection, diabetic retinopathy, macular degeneration, retrolental fibroplasia, glaucoma, and neovascular glaucoma.

Exemplary central nervous system disorders that can be treated include, but are not limited to, cortical dementias including Alzheimer's disease, neurodegeneration, and central nervous system damage resulting from stroke, ischemia, and trauma. The term “treatment” in the present context includes partial or total inhibition of dementias, including Alzheimer's disease, vascular dementia, multi-infarct dementia, pre-senile dementia, alcoholic dementia, and senile dementia.

Exemplary pain conditions that can be treated include, but are not limited to, postoperative pain, pain resulting from battle field wounds, dental pain, muscular pain, pain resulting from cancer, headaches, including sinus headache and migraine, menstrual cramps, and pain associated with inflammation.

Exemplary inflammation-related cardiovascular disorders that can be treated or prevented using compositions of the invention include, but are not limited to, vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins, and capillaries.

Exemplary angiogenesis-related disorders for which the inventive compositions are useful include, but are not limited to, inhibition of tumor angiogenesis. Such compositions also are useful for treating neoplasia, including metastasis, benign and malignant tumors, and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. The nanoparticulate nimesulide compositions of the invention can also be used to treat fibrosis that occurs with radiation therapy.

The compositions of the invention can be used to treat subjects having adenomatous polyps, including those with familial adenomatous polyposis (FAP). Additionally, such compositions can be used to prevent polyps from forming in patients at risk of FAP.

Because the nimesulide compositions of the invention inhibit prostanoid-induced smooth muscle contraction by inhibiting synthesis of contractile prostanoids, the compositions can be used in the treatment of dysmenorrhea, premature labor, asthma, and eosinophil-related disorders.

The compositions of the invention are also useful in treating indications where anti-inflammatory agents, anti-angiogenesis agents, antitumorigenic agents, immunosuppressive agents, NSAIDs, COX-2 inhibitors, analgesic agents, anti-thrombotic agents, narcotics, or antifebrile agents are typically used.

B. Surface Stabilizers

Surface stabilizers especially useful herein physically adhere on or associate with the surface of nanoparticulate nimesulide particles, but do not chemically react with the nimesulide particles or themselves. Preferably, individual molecules of the surface stabilizer are essentially free of intermolecular cross-linkages.

Combinations of more than one surface stabilizer can be used in the invention. Useful surface stabilizers include, but are not limited to, known organic and inorganic pharmaceutical excipients. Such excipients include various polymers, low molecular weight oligomers, natural products, and surfactants. Preferred surface stabilizers include nonionic, ionic, anionic, cationic, and zwitterionic surfactants.

Representative examples of surface stabilizers include hydroxypropylmethylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methyl cellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Triton X-200®, which is an alkyl aryl polyether sulfonate (Dow Chemical); Crodestas F-110°, which is a mixture of sucrose stearate and sucrose distearate (Croda Inc.); p-isononylphenoxypoly-(glycidol), also known as Olin-10G® or Surfactant 10-G® (Olin Chemicals, Stamford, Conn.); Crodestas SL-40® (Croda, Inc.); and SA9OHCO, which is C₁₈H₃₇CH₂C(O)N(CH₃)—CH₂(CHOH)₄(CH₂OH)₂ (Eastman Kodak Co.); decanoyl-N-methylglucamide; n-decyl 13-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like such as Plasdone® 5630 in a 60:40 ratio of the pyrrolidone and vinyl acetate.

More examples of useful surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexadecyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈) dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUATT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers include nonpolymeric compounds, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), which is specifically incorporated herein by reference.

Particularly preferred surface stabilizers include, but are not limited to, a copovidone, such as Plasdone® S-630 (ISP) and Kollidon® VA 64 (BASF), which are random copolymers of vinyl pyrrolidone and vinyl acetate in a 60:40 ratio, hydroxypropylmethyl cellulose, or tyloxapol.

Each of these surface stabilizers is commercially available and/or can be prepared by techniques known in the art.

C. Other Pharmaceutical Excipients

Pharmaceutical compositions of the invention may also comprise one or more binding agents, filling agents, lubricating agents, suspending agents, sweeteners, flavoring agents, preservatives, buffers, wetting agents, disintegrants, effervescent agents, and other excipients depending upon the route of administration and the dosage form desired. Such excipients are well known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCCT™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, and quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples, such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

D. Nanoparticulate Nimesulide Particle Size

Compositions of the invention contain nimesulide nanoparticles that have an effective average particle size of less than about 2000 nm (i.e., 2 microns). In preferred embodiments of the invention, the nimesulide nanoparticles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

An “effective average particle size of less than about 2000 nm” means that at least 50% of the nimesulide particles have a particle size less than the effective average, by weight, i.e., less than about 2000 nm, about 1900 nm, about 1800 nm, etc., when measured by the above-noted techniques. Preferably, at least about 70%, at least about 90%, at least about 95%, or at least about 99% of the nimesulide particles have a particle size less than the effective average, i.e., less than about 2000 nm, about 1900 nm, about 1800 nm, etc.

In the present invention, the value for D50 of a nanoparticulate nimesulide composition is the particle size below which 50% of the nimesulide particles fall, by weight. Similarly, D90 is the particle size below which 90% of the nimesulide particles fall, by weight.

If the compositions of the invention also comprise microparticulate nimesulide or non-nimesulide active agents, then the particles of such compounds have an effective average particle size greater than about 2 microns, which means that at least 50% of the particles have a size greater than about 2 microns. In other embodiments of the invention, at least about 70%, at least about 90%, at least about 95%, or at least about 99% of the nimesulide microparticulate or non-nimesulide microparticulate particles have a particle size greater than about 2 microns.

E. Concentration of Nanoparticulate Nimesulide and Surface Stabilizers

The relative amounts of at least one nimesulide and one or more surface stabilizers can vary widely. The optimal amount of the individual components depends, for example, upon physical and chemical attributes of the surface stabilizer(s) selected, such as the hydrophilic lipophilic balance (HLB), melting point, and the surface tension of water solutions of the stabilizer, etc.

Preferably, the concentration of nimesulide can vary from about 99.5% to about 0.001%, from about 95% to about 0.1%, or from about 90% to about 0.5%, by weight, based on the total combined weight of the nimesulide and at least one surface stabilizer, not including other excipients. Higher concentrations of the active ingredient are generally preferred from a dose and cost efficiency standpoint.

Preferably, the concentration of surface stabilizer can vary from about 0.5% to about 99.999%, from about 5.0% to about 99.9%, or from about 10% to about 99.5%, by weight, based on the total combined dry weight of nimesulide and at least one surface stabilizer, not including other excipients.

### VI. Methods of Making Nanoparticulate Nimesulide Compositions

Nanoparticulate nimesulide compositions can be made using any suitable method known in the art such as, for example, milling, homogenization, or precipitation techniques. Exemplary methods of making nanoparticulate compositions are described in the '684 patent. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated herein by reference.

The resultant nanoparticulate nimesulide compositions or dispersions can be utilized in solid, semi-solid, or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc. Solid dose forms of the dispersions of novel nimesulide formulations according to the present invention can be made as described in U.S. Pat. No. 6,375,986.

A. Milling to Obtain Nanoparticulate Nimesulide Dispersions

Milling nimesulide to obtain a nanoparticulate nimesulide dispersion comprises dispersing nimesulide particles in a liquid dispersion media in which the nimesulide is poorly soluble, followed by applying mechanical means in the presence of grinding media to reduce the particle size of the nimesulide to the desired effective average particle size. The dispersion media can be, for example, water, safflower oil, ethanol, t-butanol, glycerin, polyethylene glycol (PEG), hexane, or glycol. Water is a preferred dispersion media.

The nimesulide particles can be reduced in size preferably in the presence of at least one surface stabilizer. Alternatively, the nimesulide particles can be contacted with one or more surface stabilizers after attrition. Other compounds, such as a diluent, can be added to the nimesulide/surface stabilizer composition during the particle size reduction process. Dispersions can be manufactured continuously or in a batch mode.

B. Precipitation to Obtain Nanoparticulate Nimesulide Compositions

Another method of forming a nanoparticulate nimesulide composition is microprecipitation. This involves preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving nimesulide in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

C. Homogenization to Obtain Nimesulide Nanoparticulate Compositions

Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing nimesulide particles in a liquid dispersion media in which the nimesulide is poorly soluble, followed by subjecting the dispersion to homogenization to reduce the particle size of the nimesulide to the desired effective average particle size. The nimesulide particles can be reduced in size in the presence of at least one surface stabilizer. Alternatively, the nimesulide particles can be contacted with one or more surface stabilizers either before or after attrition. Other compounds, such as a diluent, can be added to the nimesulide/surface stabilizer composition either before, during, or after the size reduction process. Dispersions can be manufactured continuously or in a batch mode.

### VII. Methods of Using Nimesulide Formulations of the Invention

Nimesulide compositions of the invention can be administered to a subject via any conventional means including, but not limited to, preferably orally, vaginally, rectally, ocularly, parenterally (e.g., intravenous, intramuscular, or subcutaneous), intracisternally, pulmonary, intravaginally, intraperitoneally, topically, locally (e.g., powders, ointments or drops), or as a buccal or nasal spray. As used herein, the term “subject” is used to mean an animal, preferably a mammal, including a human or non-human. The terms patient and subject may be used interchangeably.

The present invention provides a method of prolonging plasma levels of nimesulide in a subject while achieving the desired therapeutic effect.

In one aspect, compositions of the invention are administered for treating conditions characterized by pain, inflammation, or fever. Many such conditions are set forth above.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate nimesulide compositions may also contain adjuvants such as preserving, wetting, emulsifying, and dispensing agents. Prevention of the growth of microorganisms can also be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration are preferred and include, but are not limited to, capsules, tablets, pills, powders, caplets, and granules. In such solid dosage forms, the active agent (i.e. the composition of this invention) is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable dispersions, emulsions, solutions, suspensions, syrups, and elixirs. In addition to the active agent, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Effective amounts of the inventive nimesulide compositions can be determined empirically. The compositions can be employed in pure form or, where such forms exist, in pharmaceutically acceptable salt, ester, or prodrug form. Actual dosage levels of nimesulide in the inventive nanoparticulate compositions may be varied to obtain an amount of nimesulide that is effective to obtain a desired therapeutic response for a particular composition, method of administration and condition to be treated. The selected dosage level therefore depends upon the desired therapeutic effect, the route of administration, the potency of the administered nimesulide, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

A. Treatment Applications

Nanoparticulate nimesulide compositions are useful for treating and/or preventing a wide range of conditions and disorders mediated by COX-2, including but not limited to, disorders characterized by inflammation, pain, and/or fever. Such compositions are especially useful as anti-inflammatory agents, such as in treatment of arthritis, with the additional benefit of having significantly less harmful side effects than compositions of conventional NSAIDs that lack selectivity for COX-2 over COX-1. In particular, such compositions have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs.

Thus, nanoparticulate nimesulide compositions of the invention are particularly useful as an alternative to conventional NSAIDs where such NSAIDs are contraindicated, for example in patients with peptic ulcers, gastritis, regional enteritis, ulcerative colitis, diverticulitis, or with a recurrent history of gastrointestinal lesions; gastrointestinal bleeding; coagulation disorders including anemia such as hypoprothrombinemia, hemophilia, or other bleeding problems; kidney disease; or in patients prior to surgery or patients taking anticoagulants.

Because of the rapid onset of therapeutic effect observed with the compositions of the invention, these compositions have particular advantages over prior conventional formulations for treatment of acute COX-2 mediated disorders, especially for relief of pain, for example in headache, including sinus headache and migraine.

Nimesulide is also useful in treating and/or preventing, for example, arthritic disorders, gastrointestinal conditions, inflammatory conditions, pulmonary inflammation, opthalmic diseases, central nervous systems disorders, pain, inflammation-related cardiovascular disorders, angiogenesis-related disorders, benign and malignant tumors, adenomatous polyps, disorders of the female reproductive system such as endometriosis, osteoporosis, dysmenorrhea, premature labor, asthma, eosinophil-related disorders, pyrexia, bone resorption, nephrotoxicity, hypotension, arthrosis, joint stiffness, kidney disease, liver disease including hepatitis, acute mastitis, diarrhea, colonic adenomas, bronchitis, allergic neuritis, cytomegalovirus infectivity, apoptosis including HIV-induced apoptosis, lumbago; skin-related conditions such as psoriasis, eczema, acne, burns, dermatitis, and ultraviolet radiation damage including sunburn; allergic rhinitis, respiratory distress syndrome, and endotoxin shock syndrome. Nanoparticulate nimesulide is also useful as an immunosuppressive agent.

Exemplary forms of arthritic disorders that can be treated include, but are not limited to, osteoarthritis, rheumatoid arthritis, spondyloarthropathies, gouty arthritis, juvenile arthritis, gout, ankylosing spondylitis, systemic lupus erythematosus, bursitis, tendinitis, myofascial pain, carpal tunnel syndrome, fibromyalgia syndrome, infectious arthritis, psoriatic arthritis, Reiter's syndrome, and scleroderma

Exemplary gastrointestinal conditions or ulcerative diseases that can be treated include, but are not limited to, inflammatory bowel disease, Crohn's disease, gastritis, irritable bowel syndrome, ulcerative colitis, gastric ulcer, pathological but non-malignant conditions such as hemangiomas, including infantile hemaginomas, angiofibroma of the nasopharynx, and avascular necrosis of bone.

Exemplary inflammation conditions that can be treated include, but are not limited to, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, post-operative inflammation including that following ophthalmic surgery such as cataract surgery or refractive surgery, and the like.

Exemplary pulmonary inflammation conditions that can be treated include, but are not limited to, inflammation associated with viral infections and cystic fibrosis, and in bone resorption such as that associated with osteoporosis.

Exemplary opthalmic diseases or conditions that can be treated include, but are not limited to, retinitis, conjunctivitis, retinopathies, uveitis, ocular photophobia, acute injury to the eye tissue, corneal graft rejection, ocular neovascularization, retinal neovascularization including neovascularization following injury or infection, diabetic retinopathy, macular degeneration, retrolental fibroplasia, glaucoma, and neovascular glaucoma.

Exemplary central nervous system disorders that can be treated include, but are not limited to, cortical dementias including Alzheimer's disease, neurodegeneration, and central nervous system damage resulting from stroke, ischemia, and trauma. The term “treatment” in the present context includes partial or total inhibition of dementias, including Alzheimer's disease, vascular dementia, multi-infarct dementia, pre-senile dementia, alcoholic dementia, and senile dementia.

Exemplary pain conditions that can be treated include, but are not limited to, postoperative pain, pain resulting from battle field wounds, dental pain, muscular pain, pain resulting from cancer, headaches, including sinus headache and migraine, menstrual cramps, and pain associated with inflammation.

Exemplary inflammation-related cardiovascular disorders that can be treated or prevented using compositions of the invention include, but are not limited to, vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins, and capillaries.

Exemplary angiogenesis-related disorders for which the inventive compositions are useful include, but are not limited to, inhibition of tumor angiogenesis. Such compositions also are useful for treating neoplasia, including metastasis, benign and malignant tumors, and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. The nanoparticulate nimesulide compositions of the invention can also be used to treat fibrosis that occurs with radiation therapy.

The compositions of the invention can be used to treat subjects having adenomatous polyps, including those with familial adenomatous polyposis (FAP). Additionally, such compositions can be used to prevent polyps from forming in patients at risk of FAP.

Because the nimesulide compositions of the invention inhibit prostanoid-induced smooth muscle contraction by inhibiting synthesis of contractile prostanoids, the compositions can be used in the treatment of dysmenorrhea, premature labor, asthma, and eosinophil-related disorders.

The compositions of the invention are also useful in treating indications where anti-inflammatory agents, anti-angiogenesis agents, antitumorigenic agents, immunosuppressive agents, NSAIDs, COX-2 inhibitors, analgesic agents, anti-thrombotic agents, narcotics, or antifebrile agents are typically used.

The following examples are provided to illustrate the present invention. It should be understood, however, that the invention is not to be limited to the specific conditions or details described in these examples. Throughout the specification, any and all references to a publicly available document, including a U.S. patent, are specifically incorporated by reference.

### Example 1

The purpose of this example was to prepare a nanoparticulate composition of nimesulide (Sigma, St. Louis, Mo.), a COX-2 inhibitor.

An aqueous solution of 1% (w/w) Plasdone® S-630 (International Specialty Products, Wayne, N.J.), which is a random copolymer of vinyl acetate and vinyl pyrrolidone, was prepared by dissolving 0.85 g of Plasdone® S-630 in 79.9 g of deionized water. The surface stabilizer solution was combined with 4.25 g of nimesulide (5% w/w) and PolyMill™-200 Polystyrene Milling Media (Dow Chemical, Midland, Mich.) and charged into the 150 cc batch chamber of a DYNO®-Mill Type KDL media mill (Willy Bachofen AG, Basel, Switzerland). The mill was operated for 1 hour at 4200 rpm with chilled water (10° C.) recirculated through the milling chamber.

The process yielded a colloidal dispersion of nimesulide with a mean particle size of 150 nm, a D50 of 124 nm, a D90 of 256 nm, and a D95 of 293 nm, as measured using a Horiba LA-910 Laser Scattering Particle Size Distribution Analyzer ((Horiba Instruments, Irvine, Calif.).

### Example 2

The purpose of this example was to prepare a nanoparticulate composition of nimesulide.

An aqueous solution of 1% (w/w) Plasdone® S-630 (International Specialty Products, Wayne, N.J.) and 0.2% (w/w) docusate sodium (DOSS; Cytec Industries Inc., West Paterson, N.J.) was prepared by dissolving 0.85 g of Plasdone® S-630 and 0.17 g of DOSS in 79.73 g of deionized water. The surface stabilizer solution was combined with 4.25 g of nimesulide (5% w/w) and PolyMill™-200 Polystyrene Milling Media (Dow Chemical, Midland, Mich.) and charged into the 150 cc batch chamber of a DYNO®-Mill Type KDL media mill (Willy Bachofen AG, Basel, Switzerland). The mill was operated for 2 hours at 4200 rpm with chilled water (10° C.) recirculated through the milling chamber.

The process yielded a colloidal dispersion of nimesulide with a mean particle size of 131 nm, a D50 of 111 nm, a D90 of 216 nm, and a D95 of 253 nm, as measured using a Horiba LA-910 Laser Scattering Particle Size Distribution Analyzer ((Horiba Instruments, Irvine, Calif.).

### Example 3

The purpose of this example was to prepare a nanoparticulate composition of nimesulide.

An aqueous solution of 1% (w/w) Plasdone® S-630 (International Specialty Products, Wayne, N.J.) and 0.05% (w/w) sodium lauryl sulfate (SLS) was prepared by dissolving 0.85 g of Plasdone® S-630 and 0.04 g of SLS in 79.9 g of deionized water. The surface stabilizer solution was combined with 4.25 g of nimesulide (5% w/w) and PolyMill™-200 Polystyrene Milling Media (Dow Chemical, Midland, Mich.) and charged into the 150 cc batch chamber of a DYNO®-Mill Type KDL media mill (Willy Bachofen AG, Basel, Switzerland). The mill was operated for 2 hours at 4200 rpm with chilled water (10° C.) recirculated through the milling chamber.

The process yielded a colloidal dispersion of nimesulide with a mean particle size of 116 nm, a D50 of 104 nm, a D90 of 175 nm, and a D95 of 212 nm, as measured using a Horiba LA-910 Laser Scattering Particle Size Distribution Analyzer ((Horiba Instruments, Irvine, Calif.).

### Example 4

The purpose of this example was to prepare a nanoparticulate composition of nimesulide.

An aqueous solution of 2% (w/w) hydroxypropylmethyl cellulose (HPMC, Shin Etsu) was prepared by dissolving 1.7 g of HPMC in 74.8 g of deionized water. The surface stabilizer solution was combined with 8.5 g of nimesulide (10% w/w) and PolyMill™-200 Polystyrene Milling Media (Dow Chemical, Midland, Mich.) and charged into the 150 cc batch chamber of a DYNO®-Mill Type KDL media mill (Willy Bachofen AG, Basel, Switzerland). The mill was operated for 2 hours at 4200 rpm with chilled water (10° C.) recirculated through the milling chamber.

The process yielded a colloidal dispersion of nimesulide with a mean particle size of 110 nm, a D50 of 103 nm, a D90 of 157 nm, and a D95 of 183 nm, as measured using a Horiba LA-910 Laser Scattering Particle Size Distribution Analyzer ((Horiba Instruments, Irvine, Calif.).

### Example 5

The purpose of this example was to prepare a nanoparticulate composition of nimesulide.

An aqueous solution of 2% (w/w) tyloxapol (Organichem Corp.) was prepared by dissolving 1.7 g of tyloxapol in 74.8 g of deionized water. The surface stabilizer solution was combined with 8.5 g of nimesulide (10% w/w) and PolyMill™-200 Polystyrene Milling Media (Dow Chemical, Midland, Mich.) and charged into the 150 cc batch chamber of a DYNO®-Mill Type KDL media mill (Willy Bachofen AG, Basel, Switzerland). The mill was operated for 2 hours at 4200 rpm with chilled water (10° C.) recirculated through the milling chamber.

The process yielded a colloidal dispersion of nimesulide with a mean particle size of 141 nm, a D50 of 127 nm, a D90 of 222 nm, and a D95 of 250 nm, as measured using a Horiba LA-910 Laser Scattering Particle Size Distribution Analyzer ((Horiba Instruments, Irvine, Calif.).

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A nimesulide composition comprising:
(a) particles of nimesulide or a salt thereof, wherein the particles have an effective average particle size of less than about 2000 nm; and
(b) at least one surface stabilizer adsorbed on the surface of the nimesulide particles.

2. The composition of claim 1, wherein the nimesulide is selected from the group consisting of a crystalline phase, an amorphous phase, a semi-crystalline phase, and mixtures thereof.

3. The composition of claim 1, wherein the effective average particle size of the nimesulide particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

4. The composition of claim 1, wherein the composition is formulated:
(a) for administration selected from the group consisting of oral, pulmonary, rectal, opthalmic, colonic, parenteral, intracisternal, intravaginal, intraperitoneal, local, buccal, nasal, and topical administration;
(b) into a dosage form selected from the group consisting of liquid dispersions, oral suspensions, gels, aerosols, ointments, creams, controlled release formulations, fast melt formulations, lyophilized formulations, tablets, capsules, delayed release formulations, extended release formulations, pulsatile release formulations, and mixed immediate release and controlled release formulations; or
(c) any combination thereof.

5. (canceled)

6. The composition of claim 1, wherein the composition further comprises one or more pharmaceutically acceptable excipients, carriers, or a combination thereof.

7. The composition of claim 1, wherein:
(a) the nimesulide or a salt thereof is present in an amount selected from the group consisting of from about 99.5% to about 0.001%, from about 95% to about 0.1%, and from about 90% to about 0.5%, by weight, based on the total combined dry weight of the nimesulide or a salt thereof and at least one surface stabilizer, not including other excipients;
(b) the at least one surface stabilizer is present in an amount selected from the group consisting of from about 0.5% to about 99.999% by weight, from about 5,0% to about 99.9% by weight, and from about 10% to about 99.5% by weight, based on the total combined dry weight of the nimesulide or a salt thereof and at least one surface stabilizer, not including other excipients; or
(c) any combination thereof.

8. (canceled)

9. The composition of claim 1, comprising two or more surface stabilizers.

10. The composition of claim 1, wherein the surface stabilizer is selected from the group consisting of an anionic surface stabilizer, a cationic surface stabilizer, a zwitterionic surface stabilizer, a non-ionic surface stabilizer, and an ionic surface stabilizer.

11. The composition of claim 10, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, lose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, and random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipid, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quanternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl hydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxymethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, quaternized hydroxyethyl cellulose, tetrabutylammonium bromide, benzyl trimethylammonium bromide, choline esters, benzalkonium chloride, stearalkonium chloride compounds cetyl pyridinium bromide, cetyl pyridinium chloride, halide salts of quaternized polyoxyethtlalkylamines, quaternary ammonium compounds, alkly pyridinium salts; amines, amine amine salts, amine oxides, imide azolinium salts, protonated quaternary acrylamides, methylated quaternary polymers, and cationic guar.

12-13. (canceled)

14. The composition of claim 1, comprising as a surface stabilizer a random copolymer of vinyl acetate and vinyl pyrrolidone, hydroxypropylmethyl cellulose, or tyloxapol.

15. The composition of claim 10, wherein the composition is bioadhesive.

16. The composition of claim 1, wherein the Tₘₐₓ:
(a) of the nimesulide, when assayed in the plasma of a mammalian subject following administration, is less than the Tₘₐₓ for a conventional, non-nanoparticulate form of nimesulide, administered at the same dosage;
(b) is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, and not greater than about 10% of the Tₘₐₓ, exhibited by a non-nanoparticulate formulation of nimesulide, administered at the same dosage; or
(c) any combination thereof.

17. (canceled)

18. The composition of claim 1, wherein the Cₘₐₓ:
(a) of the nimesulide, when assayed in the plasma of a mammalian subject following administration, is greater than the Cₘₐₓ for a conventional, non-nanoparticulate form of nimesulide, administered at the same dosage;
(b) is selected from the group consisting of at least about 10%, at least about 20%, at least about at least about 40% at least about 50% at least about 60%, at least about 70% at least about 80 at least about 90% and at least about 100% greater than the Cₘₐₓ exhibited by a non-nanoparticulate formulation of nimesulide, administered at the same dosage; or
(c) any combination thereof.

19. (canceled)

20. The composition of claim 1, wherein the AUC:
(a) of the nimesulide, when assayed in the plasma of a mammalian subject following administration, is greater than the AUC for a conventional, non-nanoparticulate form of nimesulide, administered at the same dosage;
(b) is selected from the group consisting of at least about 10%, at least about 20%, at least about 30%, at least about 40%, at least about 50%, at least about 60%, at east about 70%, at least about 80%, at least about 90%, and at least about 100% greater than the AUC exhibited by a non-nanoparticulate formulation of nimesulide, administered at the same dosage.

21. (canceled)

22. The composition of claim 1:
(a) which does not produce significantly different absorption levels when administered under fed as compared to fasting conditions;
(b) wherein the difference in absorption of the nimesulide composition of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25% less than about 20%, less than about 15%, less than about 10%, less than about 5%, and less than about 3%; or
(c) any combination thereof.

23. (canceled)

24. The composition of claim 1, wherein administration of the composition to a subject in a fasted state is bioequivalent to administration of the composition to a subject in a fed state, when administered to a human.

25. The composition of claim 24, wherein “bioequivalency”
(a) is established by a 90% Confidence Interval of between 0.80 and 1.25 for both Cₘₐₓ and AUC, when administered to a human; or
(b) is established by a 90% Confidence Interval of between 0.80 and 1.25 for AUC and a 90% Confidence Interval of between 0.70 to 1.43 for Cₘₐₓ, when administered to a human.

26. (canceled)

27. The composition of claim 1, further comprising at least one additional nimesulide composition having an effective average particle size which is different that the effective average particle size of the nimesulide composition of claim 1.

28. The composition of claim 1, wherein:
(a) upon administration the composition redisperses such that the nimesulide particles have an effective average particle size of less than about 2000 nm;
(b) upon administration the composition redisperses such that the nimesulide particles have an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm less than about 1000 nm, less than about 900 nm, less than about 800 nm, less than about 700 nm, less than about 600 nm less than about 500 nm less than about 400 nm, less than about 300 nm, less than about 250 nm, less than about 200 nm, less than about 150 nm less than about 100 nm, less than about 75 nm, and less than about 50 nm;
(c) the composition redisperses in a biorelevant media such that the nimesulide particles have an effective average particle size of less than about 2 microns;
(d) the composition redisperses in a biorelevant media such that the nimesulide particles have an effective average particle size selected from the group consisting of less than about 1900 nm, less than about 1800 nm, less than about 1700 nm, less than about 1600 nm less than about 1500 nm, less than about 1400 nm less than about 1300 nm less than about 1200 nm less than about 1100 nm less than about 1000 nm, less than about 900 nm, less than about 800 nm less than about 700 nm, less than about 600 nm, less than about 500 nm, less than about 400 nm less than about 300 nm, less than about 250 nm less than about 200 nm, less than about 150 nm less than about 100 nm, less than about 75 nm and less than about 50 nm, or
(e) any combination thereof.

29-31. (canceled)

32. The composition of claim 1 formulated into a liquid dosage form, wherein the dosage form:
(a) has a viscosity of less than about 2000 mPa·s, measured at 20° C., at a shear rate of 0.1 (1/s):
(b) has a viscosity at a shear rate of 0.1 (1/s) selected from the group consisting of from about 2000 mPa·s to about 1 mPa·s, from about 1900 mPa·s to about 1 mPa·s, from about 1800 mPa·s to about 1 mPa·s, from about 1700 mPa·s to about 1 mPa·s, from about 1600 mPa·s to about 1 mPa·s, from about 1500 mPa·s to about 1 mPa·s, from about 1400 mPa·s to about 1 mPa·s, from about 1300 mPa·s to about 1 mPa·s, from about 1200 mPa·s to about 1 mPa·s, from about 1100 mPa·s to about 1 mPa·s, from about 1000 mPa·s to about 1 mPa·s, from about 900 mPa·s to about 1 mPa·s, from about 800 mPa·s to about 1 mPa·s, from about 700 mPa·s to about 1 mPa·s, from about 600 mPa·s to about 5 mPa·s from about 500 mPa·s to about 1 mPa·s, from about 400 mPa·s to about 1 mPa·s from about 300 mPa·s to about 1 mPa·s from about 200 mPa·s to about 1 mPa·s, from about 175 mPa·s to about 1 mPa·s from about 150 mPa·s to about 1 mPa·s, from about 125 mPa·s to about 1 mPa·s, from about 100 mPa·s to about 1 mPa·s, from about 75 mPa·s to about 1 mPa·s, from about 50 mPa·s to about 1 mPa·s from about 25 mPa·s to about 1 mPa·s, from about 15 mPa·s to about 1 mPa·s, from about 10 mPa·s to about 1 mPa·s, and from about 5 mPa·s to about 1 mPa·s:
(c) has a viscosity selected from the group consisting of less than about 1/200, less than about 1/100, less than about 1/50, less than about 1/25, and less than about 1/10 of the viscosity of a liquid dosage form of conventional non-nanoparticulate nimesulide at about the same concentration per ml of nimesulide;
(d) has a viscosity selected from the group consisting of less than about 5%, less than about 10% less than about 15%, less than about 20%, less than about 25%, less than about 30%, less than about 35%, less than about 40%, less than about 450%, less than about 50%, less than about 55%, less than about 60%, less than about 65%, less than about 70%, less than about 75%, less than about 80%, less than about 85%, and less than about 90% of the viscosity of a liquid dosage form of conventional, non-nanoparticulate nimesulide at about the same concentration per ml of nimesulide; or
(e) any combination thereof.

33-35. (canceled)

36. The composition of claim 1, additionally comprising one or more non-nimesulide active agents.

37. The composition of claim 36, wherein said non-nimesulide active agent is:
(a) selected from the group consisting of an analgesic, an anti-inflammatory, an antipyretic, and a vasomodulator;
(b) selected from the group consisting of nutraceuticals, proteins, peptides, nucleotides, amino acids, anti-obesity drugs, central nervous system stimulants, carotenoids, corticosteroids, elastase inhibitors, anti-fungals, oncology therapies, anti-emetics, analgesics cardiovascular agents, anti-inflammatory agents, NSAIDs, non-nimesulide COX-2 inhibitors, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, alpha-adrenergic receptor blocking agents, beta-adrenoceptor blocking agents, blood products and substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin and biphosphonates, prostaglandins, radio-pharmaceuticals, sex hormones, anti-allergic agents, stimulants and anoretics, sympathomimetics, thyroid agents, vasodilators, vasomodulators, and xanthines;
(c) is a nutraceutical selected from the group consisting of lutein, folic acid, fatty acids, fruit extracts, vegetable extracts, vitamin supplements, mineral supplements, phosphatidylserine, lipoic acid, melatonin, glucosamine/chondroitin, Aloe Vera, Guggul, glutamine, amino acids, green tea, lycopene, whole foods, food additives, herbs, phytonutrients, antioxidants, flavonoid constituents of fruits, evening primrose oil, flax seeds, fish oils, marine animal oils, and probiotics;
(d) selected from the group consisting of aceclofenac, acemetacin, e-acetamidocaproic acid, acetaminophen, acetaminosalol, acetanilide, acetylsalicylic acid S-adenosylmethionine, alclofenac, alfentanil, allylprodine, alminoprofen, aloxiprin, alphaprodine aluminum bis(acetylsalicylate), amfenac, aminochlorthenoxazin, 3-amino-4-hydroxybutyric acid, 2-amino-4-picoline, aminopropylori, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol bromfenae, p-bromoacetanilide, 5-bromosalicylic acid acetate, bromosaligenin, bucetin, bucloxic acid, bucolome, bufexamac, bumadizon, buprenorphine butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol dimethylthiambutene, dioxaphetyl butyrate, dipipanone, diprocetyl, dipyrone, ditazol, droxicam, emorfazone, enfenamic acid, epirizole, eptazocine etersalate, ethenzamide, ethoheptazine, ethoxazene, ethylmethythiambutene, ethylmorphine, etodolac, etofenamate etonitazene eugenol, febinac, fenbufen, fenclozic acid, fedosal, fenoprofen, fentanyl, fentiazac, fepradinol, feprazone, floctafenine, flufenamic acid, flunoxapofen, fluoresone, flupirtine, fluproquazone flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidin, phenoprazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remicfentanil, rimazolium metilsulfate, salacetamide, salicin, salicylamide salicylamide o-acetic acid, salicylsulfuric acid salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide tilidine tinoridine, tolfenamnic acid, tolmetin tramadol, tropesin, viminol, xenbucin, ximonrofen, zaltoprofen, and zomepirac;
(e) is a vasomodulator selected from the group consisting of caffeine, theobromine, and theophylline;
(f) is an NSAID selected from the group consisting of nabumetone, tiaramide, proquazone, bufexamac, flumizole, epirazole, tinoridine, timegadine, dapsone, aspirin, difunisal, benorylate, fosfosal, diclofenac, alclofenac, fenclofenac, etodolac, indomethacin, sulindac, tolmetin, fentiazac, tilomisole, carprofen, fenbufen, flurbiprofen, ketoprofen, oxaprozin, suprofen, tiaprofenic acid, ibuprofen, naproxen, fenoprofen, indoprofen, pirprofen, flufenamic, mefenamic, meclofenamic, niflumic, oxyphenbutazone, phenylbutazone, apazone feprazone, piroxicam, sudoxicam, isoxicam, and tenoxicam; or
(g) is a COX-2 inhibitor selected from the group consisting of celecoxib, rofecoxib, meloxicam, valdecoxib, parecoxib, etoricoxib, SC-236, NS-398, SC-58125, SC-57666, SC-558, SC-560, etodolac, DFU, monteleukast, L-745337, L-761066, L-761000, L-748780, DUP-697, PGV 20229, iguratimod, BF 389, CL 1004, PD 136005, PD 142893, PD 138387, PD 145065, flurbiprofen, nabumetone, flosulide, piroxicam, diclofenac, lumiracoxib, D 1367, R 807, JTE-522, FK-3311, FK 867, FR 140423, FR 115068, GR 253035, RWJ 63556, RWJ 20485, ZK 38997, S 2474, zomepirac analogs, RS 104894, SC 41930, pranlukast, SB 209670, and APHS.

38-43. (canceled)

44. The composition of claim 1, which has been sterile filtered.

45. A method of making a nimesulide composition comprising contacting particles of nimesulide or a salt thereof with at least one surface stabilizer for a time and under conditions sufficient to provide a nimesulide composition having an effective average particle size of less than about 2000 nm and having the at least one surface stabilizer adsorbed on surface of nimesulide particles.

46. The method of claim 45, wherein said contacting comprises grinding, wet grinding, homogenizing, solvent precipitation, or any combination thereof.

47-62. (canceled)

63. A method of treating a subject in need comprising administering to the subject an effective amount of a composition comprising:
(a) particles of nimesulide or a salt thereof, wherein the nimesulide particles have an effective average particle size of less than about 2000 nm; and
(b) at least one surface stabilizer adsorbed on the surface of the nimesulide particles.

64-93. (canceled)

93. The method of claim 63, wherein the subject is a human.

94. The method of claim 63, wherein the method is used to treat a condition selected from the group consisting of rheumatic and joint diseases, arthritis, rheumatoid arthritis, osteoarthritis, periarthritis, tendonitis, bursitis, ankylosing spondylitis, joint stiffness, lower back pain, gynecological conditions, menstrual migraine attack, dysmenorrhoea, pelvic inflammatory disease, urological conditions, urethritis, prostatitis, and vesiculitis pyrexia, cardiovascular diseases, atherosclerosis, hypotension, thrombophlebitis, arthrosis; inflammatory conditions, otitis, rhinitis, sinusitis, pharyngitis, bronchitis nephrotoxicity, mastitis, asthma, cancer, trauma, surgery, migraine headaches, kidney disease, Alzheimer's disease, familial adenomatous polyposis, diarrhea, colonic adenomas bone resorption, and related conditions.

95. The method of claim 63, wherein the method is used to treat a condition where anti-inflammatory agents, anti-angiogenesis agents, antitumorigenic agents, immunosuppressive agents, NSAIDs, COX-2 inhibitors, analgesic agents, anti-thrombotic agents, narcotics, or antifebrile agents are typically used.
